(19) United States
(12) Patent Application Publication
(10) Pub. No.: US 2005/0148889 A1
(43) Pub. Date: Jul. 7, 2005

(54) Wristwatch-typed heartbeat sensing device with two pairs of contacts

(76) Inventor: Yu-Yu Chen, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/747,408

(22) Filed: Dec. 30, 2003

(51) Int. Cl. A61B 5/0402

(52) U.S. Cl. 600/509

(57) ABSTRACT

A wristwatch-typed heartbeat sensing device includes a casing which defines a hollow space for the mounting of a circuit board, a pair of top conductive contacts which include a first top conductive contact and a second top conductive contact, the first and second top conductive contacts separately mounted on the top surface of the casing and respectively connected to a control circuit of the circuit board, a pair of bottom conductive contacts which include a first bottom conductive contact and a second bottom conductive contact, the first and second bottom conductive contacts separately mounted at the bottom cover of the casing and respectively connected to the control circuit. When the heartbeat sensing device is fastened on the wrist of a user by means of a watch band, the first and second bottom conductive contacts contact the user's wrist and generate a first heartbeat signal. When the user touches the first and second top conductive contacts, a second heartbeat signal is generated. The two heartbeat signals are transmitted to the control circuit in order to generate a heartbeat data of the user.

### BACKGROUND OF THE INVENTION

1. Field of the Invention

The present invention relates to a heartbeat sensing device, especially to a wristwatch-typed heartbeat sensing device possessing two pairs of contacts.

2. Description of the Prior Art

Heartbeat sensing device is a device for detecting and displaying the heartbeats of a user. It has been widely applied for medical diagnosis and sporting purposes. By means of the heartbeat sensing device, the heartbeats are detected and displayed, thereby the body status of the user or patient can be monitored. Practically, a heartbeat sensing device is commonly installed to work in coordination with an exercise equipment. The heartbeats of the exerciser are detected and shown via a display unit of the heartbeat sensing device. Moreover, the heartbeat sensing device may be equipped with other devices like audio device, alarm and so on, so as to provide the user with multiple practical functions.

Basically, a heartbeat sensing device comprises a rubber conductor which is an electrode capable of detecting the heartbeat signals and a control circuit for processing the heartbeat signals. The rubber conductor is electrically connected to the control circuit, and the heartbeat signals detected by the rubber conductor are transmitted to the processing circuit of the control circuit for processing and then displayed on the display unit.

For a wireless heartbeat sensing device, besides the rubber conductor and control circuit, it further comprises a wireless transmitting circuit which emits the detected heartbeat signals wirelessly to a remote receiver which is positioned within an effective distance from the heartbeat sensing device. The remote receiver receives and displays the signals.

Currently, various types of heartbeat sensing device are available in the market. Some of the heartbeat sensing devices is in the wristwatch form. The wristwatch-typed heartbeat sensing device is fitted on the wrist of the user for detecting the heartbeats.

In the operation of the heartbeat sensing device, good electrical connection between the rubber connector and the control circuit is critical for effective and precise transmission of the heartbeat signals. In the past few years, the manufacturers have been aimed and devoted to develop a good electrical connection that precisely transmits the heartbeat signals with high fidelity.

The electrical connection of the conventional heartbeat sensing device generally comprises a first contact at the top surface of the casing and a second contact at the bottom surface of the casing to get in touch with the skin of the user. Please refer to FIG. 1 which shows a perspective view of a conventional wristwatch-typed heartbeat sensing device. As shown, the heartbeat sensing device 100a comprises a display device 10a and a first conductive contact 10b at the top surface of the casing. A second conductive contact (not shown) is formed at the bottom surface of the casing of the heartbeat sensing device 100a. When the wristwatch-typed heartbeat sensing device 100a is put on the wrist of the user, the second conductive contact directly contacts the user's wrist. When the user touches the first conductive contact 10b with his other hand, the control circuit inside the heartbeat sensing device 100a detects the heartbeat of the user and transmits a signal to the display device 10a for displaying. However, such a wristwatch-typed heartbeat sensing device has many drawbacks, including poor electrical connection, weak heartbeat signal, low fidelity and low precision caused by poor contact between the second conductive contact and the user's wrist, surface staining, wetting, abrasion and vibration. Those problems frequently happen in wristwatch-typed heartbeat sensing device, and affect the normal operation and shorten the service life of heartbeat sensing device.

### SUMMARY OF THE INVENTION

Consequently, a primary object of the present invention is to provide a wristwatch-typed heartbeat sensing device which has two pairs of contacts. One pair of contacts is formed on the top surface of the heartbeat sensing device and the other pairs of contacts is formed at the bottom surface of the heartbeat sensing device. Thereby, the heartbeats of the user can be precisely detected.

Another object of the present invention is to provide a wristwatch-typed heartbeat sensing device which is simple in structure. The two pairs of conductive contacts are connected to the control circuit of the heartbeat sensing device by means of conductive poles or wires.

To achieve the above objects, in accordance with the present invention, there is provided a wristwatch-typed heartbeat sensing device including a casing which defines a hollow space for the mounting of a circuit board, a pair of top conductive contacts which include a first top conductive contact and a second top conductive contact, the first and second top conductive contacts separately mounted on the top surface of the casing and respectively connected to a control circuit of the circuit board, a pair of bottom conductive contacts which include a first bottom conductive contact and a second bottom conductive contact, the first and second bottom conductive contacts separately mounted at the bottom cover of the casing and respectively connected to the control circuit. When the heartbeat sensing device is fastened on the user's wrist by means of a watch band, the first and second bottom conductive contacts contact the user's wrist and generate a first heartbeat signal. When the user touches the first and second top conductive contacts, a second heartbeat signal is generated. The two heartbeat signals are transmitted to the control circuit in order to generate a heartbeat data of the user.

### BRIEF DESCRIPTION OF THE DRAWINGS

The present invention will be apparent to those skilled in the art by reading the following description of preferred embodiments thereof, with reference to the attached drawings, in which:

FIG. 1 is a perspective view of a conventional wristwatch-typed heartbeat sensing device;

FIG. 2 is a perspective view of a wristwatch-typed heartbeat sensing device constructed in accordance with a preferred embodiment of the present invention;

FIG. 3 is a top view of the wristwatch-typed heartbeat sensing device of the present invention, showing a first top conductive contact and a second top conductive contact;

FIG. 4 is a bottom view of the wristwatch-typed heartbeat sensing device of the present invention, showing a first bottom conductive contact and a second bottom conductive contact;

FIG. 5 is a front side view of the wristwatch-typed heartbeat sensing device of the present invention, showing a connection of a watch band to a casing of the heartbeat sensing device;

FIG. 6 is a left side view of the wristwatch-typed heartbeat sensing device of the present invention, showing the connection of the watch band to the casing of the heartbeat sensing device;

FIG. 7 is a sectional view taken along line 7-7 of FIG. 3; and

FIG. 8 is a block diagram of a control circuit of the wristwatch-typed heartbeat sensing device of the present invention.

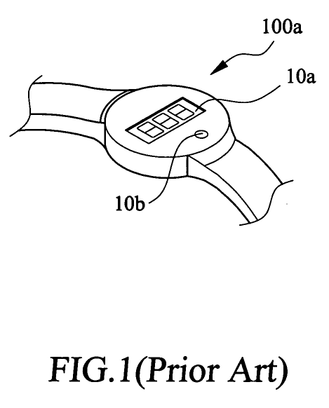

### DETAILED DESCRIPTION OF THE PREFERRED EMBODIMENTS

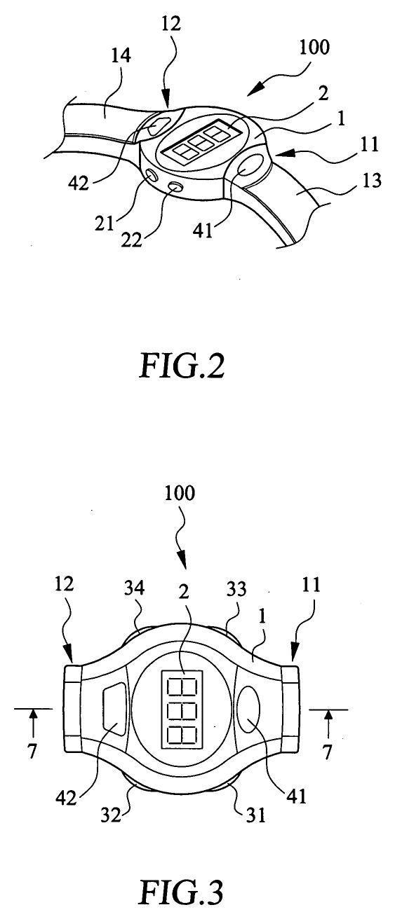

With reference to the drawings and in particular to FIG. 2, a wristwatch-typed heartbeat sensing device constructed in accordance with a preferred embodiment of the present invention is shown. The wristwatch-typed heartbeat sensing device 100 comprises a casing 1 which is configured in the form of a wristwatch having a first end 11 and a second end 12 opposite to the first end 11. A display 2 is arranged on the top surface of the casing 1 that may be a LCD display for displaying the heartbeats of the user.

The two lateral sides of the casing 1 are mounted with a plurality of control buttons for setting of parameters and controlling the functions of the heartbeat sensing device 100. Please also refer to FIG. 3 which is a top view of the heartbeat sensing device. For example, a set button 31 is provided for setting of various parameters of the heartbeat sensing device 100 including upper limited alarm value for heartbeat rate, time and so on.

Furthermore, the casing 1 is provided with a switch button 32 for switching the operation modes of the heartbeat sensing device 100. The operation modes include time display mode, heartbeat sensing mode and so on. A start/stop button 33 is also provided on the casing 1 for starting up or stopping the detection of heartbeat.

Moreover, a backlight control button 34 is provided on the casing 1 for turning on/off a backlight of the heartbeat sensing device 100. The backlight may be generated by a lighting element disposed in a hollow space of the casing 1 for providing lighting.

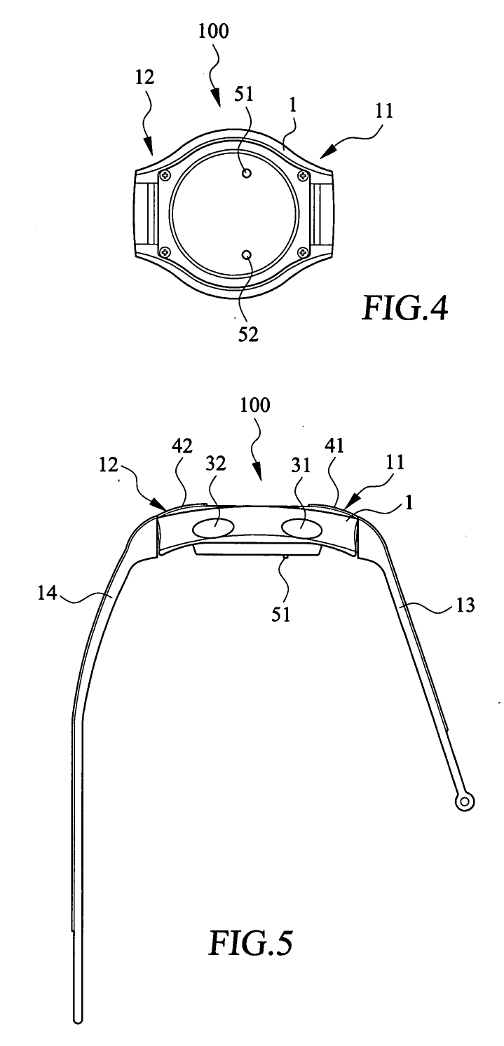

Please also refer to FIG. 4 which is a bottom view of the heartbeat sensing device. A first top conductive contact 41 and a second top conductive contact 42 are arranged on the top surface of the casing 1, and a first bottom conductive contact 51 and a second bottom conductive contact 52 are arranged at the bottom surface of the casing 1. Preferably, the distance between the first bottom conductive contact 51 and the second bottom conductive contact 52 is larger than 12 mm.

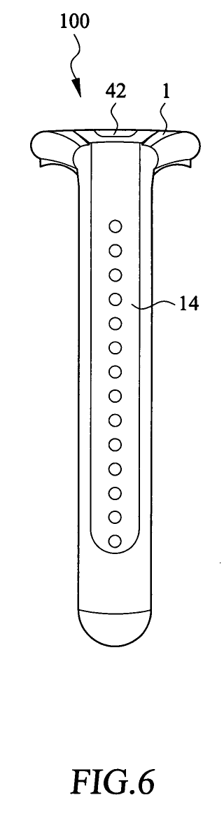

The first and second ends 11, 12 of the casing 1 are connected respectively with two straps 13, 14 of a watch band for fastening the heartbeat sensing device 100 to a user's wrist. FIG. 5 is a front side view showing the connection of the straps 13, 14 of the watch band to the casing 1 of the heartbeat sensing device 100. FIG. 6 is a left side view showing the connection of the straps 13, 14 of the watch band to the casing 1.

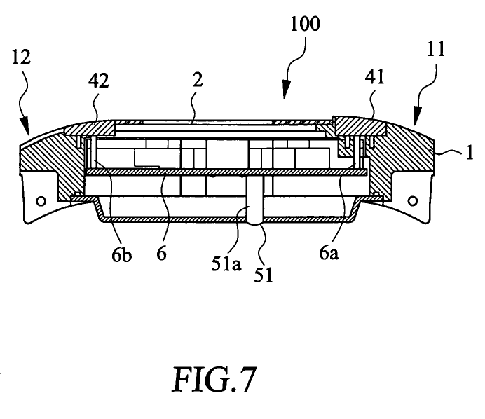

Please refer to FIG. 7 which is a sectional view taken along line 7-7 of FIG. 3. As shown, the casing 1 defines an internal hollow space for receiving a circuit board 6 and other electrical components like integrated circuit and battery. The circuit board 6 comprises a control circuit 60 for controlling the operation of the heartbeat sensing device 100. A pair of conductive poles 6a, 6b are erected from the upper surface of the circuit board 6 and extended to connect respectively to the first and second top conductive contacts 41, 42. Also, the first and second bottom conductive contacts 51, 52 are extended from the bottom surface of the circuit board 6.

The electrical connections between the conductive contacts 41, 42, 51, 52 and the circuit board 6 are accomplished by electric welding. Other measures by e.g. wiring, plugging of pins, press contact and so on may also be applied to provide electrical connections.

In the preferred embodiment, the first bottom conductive contact 51 is formed of a conductive pole 51a extending from the bottom of the circuit board 6. The top end of the conductive pole 51a is electrically connected to the circuit board 6 and the bottom end of the conductive pole 51a is protruded slightly outwardly from the bottom cover 15 of the heartbeat sensing device 100 to form the first bottom conductive contact 51.

When the wristwatch-typed heartbeat sensing device 100 is fastened to the wrist of the user by means of the straps 13, 14, the first and second bottom conductive contacts 51, 52 get in touch with the user's wrist. The user can touch the first and second top conductive contacts 41, 42 with his other hand. In other words, the wristwatch-typed heartbeat sensing device 100 is able to detect the user's heartbeats through the four conductive contacts 41, 42, 51, 52. Accordingly, when the user touches the wristwatch-typed heartbeat sensing device 100 with his hands, two heartbeat signals are generated respectively by the first top conductive contact 41 and the second top conductive contact 42, and the first bottom conductive contact 51 and the second bottom conductive contact 52. The two heartbeat signals are transmitted to the control circuit 60 is order to produce a heartbeat data of the user.

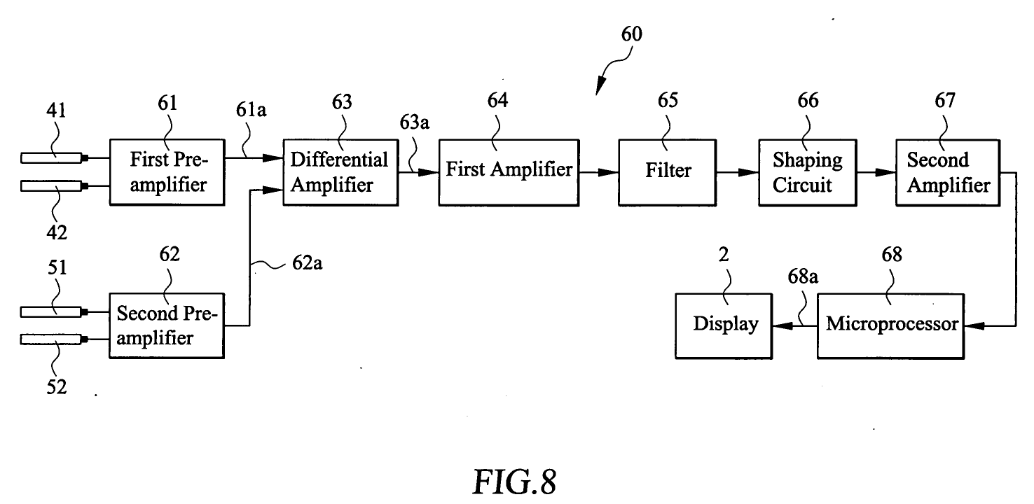

FIG. 8 shows a block diagram of the control circuit of the present invention. The control circuit 60 comprises a first pre-amplifier 61, a second pre-amplifier 62, a differential amplifier 63, a first amplifier 64, a filter 65, a shaping circuit 66, a second amplifier 67 and a microprocessor 68.

The first pre-amplifier 61 comprises two input terminals, in which a first input terminal is connected to the first top conductive contact 41 and the second input terminal is connected to the second top conductive contact 42. When the user touches the first and second top conductive contacts 41, 42 with his hand, a first differential signal 61a is generated.

The second pre-amplifier 62 comprises two input terminals, in which a first input terminal is connected to the first bottom conductive contact 51 and the second input terminal is connected to the second bottom conductive contact 52. When the user touches the first and second bottom conductive contacts 51, 52 with his hand, a second differential signal 62a is generated.

The differential amplifier 63 comprises two input terminals. A first input terminal is connected to the first pre-amplifier 61 for receiving the first differential signal 61a, while a second input terminal is connected to the second pre-amplifier 62 for receiving the second differential signal 62a. The differential amplifier 63 generates a differential output signal 63a based on the difference between the first differential signal 61a and the second differential signal 62a.

The differential output signal 63a is transmitted to the first amplifier 64 for amplifying. The amplified signal is then transmitted to the filter 65 to filter the noise. The filtered signal is forwarded to the shaping circuit 66 for signal waveform shaping, and then to the second amplifier 67 for further amplifying.

The amplified signal is subsequently forwarded from the second amplifier 67 to the microprocessor 68 for processing and calculation. The microprocessor 68 generates a heartbeat data 68a to the display 2 for displaying.

With the embodiment described above, it is understood that the present invention provides an improved wristwatch-typed heartbeat sensing device that can precisely detect the user's heartbeat and eliminate the problem of low fidelity or weak heartbeat signal. As a whole, it is practical for use and superior to similar products available in the market.

Although the present invention has been described with reference to the preferred embodiments thereof, it is apparent to those skilled in the art that a variety of modifications and changes may be made without departing from the scope of the present invention which is intended to be defined by the appended claims.

1. A wristwatch-typed heartbeat sensing device with two pairs of contacts for detecting heartbeat signals of a user, comprising:
a casing, which defines an internal hollow space and comprises a first end, a second end, a watch band with two straps respectively connected to the first end and the second end, and a circuit board which is mounted in the hollow space of the casing, a control circuit being arranged on the circuit board;
a pair of top conductive contacts, comprising a first top conductive contact and a second top conductive contact separately and respectively formed at a top surface of the casing and electrically connected to the circuit board; and
a pair of bottom conductive contacts, comprising a first bottom conductive contact and a second bottom conductive contact separately and respectively formed at a bottom surface of the casing and electrically connected to the circuit board;
wherein when the casing is put on a wrist of one hand of the user by means of the straps, the first and second bottom conductive contacts contact the wrist of the user and generate a first heartbeat signal, and when the user touches the first and second top conductive contacts with his other hand, a second heartbeat signal is generated, and the first and second heartbeat signals are transmitted to the control circuit of the circuit board so as to generate a heartbeat data of the user based on the first and second heartbeat signals.

2. The wristwatch-typed heartbeat sensing device as claimed in claim 1, wherein the first and second bottom conductive contacts are formed of conductive poles extending from the bottom of the circuit board and protruded slightly outwardly from the bottom of the casing.

3. The wristwatch-typed heartbeat sensing device as claimed in claim 1, wherein the casing further comprises a display which is mounted at the top surface of the casing, the display electrically being connected to the circuit board for displaying the heartbeat data of the user.

4. The wristwatch-typed heartbeat sensing device as claimed in claim 1, wherein the control circuit comprises:
a first pre-amplifier, comprising a first input terminal which is connected to the first top conductive contact and a second input terminal which is connected to the second top conductive contact to generate a first differential signal from the first heartbeat signal detected by the first and second top conductive contacts;
a second pre-amplifier, comprising a first input terminal which is connected to the first bottom conductive contact and a second input terminal which is connected to the second bottom conductive contact to generate a second differential signal from the second heartbeat signal detected by the first and second bottom conductive contacts;
a differential amplifier, comprising two differential signal input terminals, in which the first input terminal is connected to the first pre-amplifier for receiving the first differential signal and the second input terminal is connected to the second pre-amplifier for receiving the second differential signal, and generating a differential output signal based on the difference between the first differential signal and the second differential signal;
a microprocessor, which receives the differential output signal from the differential amplifier, calculates and generates a heartbeat data; and
a display, which receives the heartbeat signal from the microprocessor and displays the heartbeat data.

5. The wristwatch-typed heartbeat sensing device as claimed in claim 4, wherein the control circuit further comprises a filter which is connected between the differential amplifier and the microprocessor.

6. The wristwatch-typed heartbeat sensing device as claimed in claim 5, wherein the control circuit further comprises a shaping circuit which is connected between the filter and the microprocessor.
